(19) United States
(12) Patent Application Publication
(10) Pub. No.: US 2022/0022759 A1
(43) Pub. Date: Jan. 27, 2022

(54) A METHOD OF DETECTING A FLOW IN A SEQUENCE OF IMAGES

(71) Applicant: The University of Western Australia, Crawley, Western Australia (AU)

(72) Inventors: Peijun Gong, Leeming, Western Australia (AU); Qiang Wang, Nedlands, Western Australia (AU); David D. Sampson, Millbrook, Guildford Surrey (AU)

(21) Appl. No.: 17/292,506

(22) PCT Filed: Nov. 4, 2019
(86) PCT No.: PCT/AU2019/051211

§ 371 Date: May 10, 2021

(30) Foreign Application Priority Data
Nov. 8, 2018 (AU) 2018904266

(51) Int. Cl. A61B 5/026; G06T 7/262

(52) U.S. Cl. CPC A61B 5/026; G06T 7/262; G06T 2207/20056; G06T 2207/30088; G06T 2207/10101; G06T 2207/30041; G06T 2207/30104

(57) ABSTRACT

A method of detecting a flow in sequence of images of a material. Providing a sequence of at least three images of an area of the material. Each image includes a plurality of voxels or regions of interest such that the at least three images of the area of the material provide for each voxel or region of interest an intensity for at least three points in time, Fourier transforming for each voxel or region of interest to obtain a frequency distribution including the intensities for the at least three points in time, analysing for each voxel or region of interest and generating a processed image of area of the material including the voxels or regions of interest, associating voxels or regions of interest that have a larger amplitude at a higher frequency range with a first visual property and voxels or regions of interest that have smaller amplitude in the higher frequency range with a second visual property.

### FIELD OF THE INVENTION

The present invention relates generally to a method of detecting a flow, such as a flow of blood within a blood vessel, in a sequence of images, which may be images of biological tissue. The present invention relates for example, though is not limited to, a method of processing an optical coherence tomography (OCT) image of tissue in order to improve the contrast of blood vessels.

### BACKGROUND

As an extension of optical coherence tomography (OCT), optical coherence tomography angiography (OCTA) provides a non-invasive technique for imaging tissue vasculature, such as small blood vessels, including arterioles, capillaries and venules. While the image contrast in OCT is determined by the level of backscattering in the tissue, OCTA allows imaging the microvascular network via motion-induced changes in the OCT signal. OCTA typically allows achieving an image resolution and field of view in the ranges of 2-20 μm and a few mm to ˜20 mm, respectively. The imaging depth may be limited to less than 1 mm for human tissue.

OCTA identifies blood vessels by identifying differences in the OCT signal of time between that arising from moving scatterers in blood and that due to the surrounding largely static tissue. Such flow-induced differences are encoded in both the amplitude and phase of the complex OCT signal and may be detected by quantifying temporal changes in the OCT amplitude signal using speckle variance and/or correlation mapping/speckle decorrelation analyses.

There is however a need in OCTA to improve the sensitivity of detection of small blood vessels with low flow contrast, as well as to extend the imaging depth and field of view so as to provide the capability to image deeper blood vessels and larger tissue areas. Further, it would also be advantageous if processing speeds of images in OCTA could be improved.

### SUMMARY OF THE INVENTION

In accordance with a first aspect of the invention, there is provided a method of detecting a flow in sequence of images of a material, the method comprising the steps of:

providing a sequence of at least three images of an area of the material, each image including a plurality of voxels or regions of interest such that the at least three images of the area of the material provide for each voxel or region of interest an intensity I(t) as a function of time t for at least three points in time;

Fourier transforming I(t) for each voxel or region of interest to obtain a distribution I(ω) of frequency co, I(t) including the intensities for the at least three points in time and; and

analysing I(ω) for each voxel or region of interest and generating a processed image of area of the material including the voxels or regions of interest, comprising associating voxels or regions of interest that have a larger amplitude I_{L}(ω_{H}) at a frequency ω_{H} in a higher frequency range with a first visual property and voxels or regions of interest that have smaller amplitude I_{S}(ω_{H}) in the higher frequency range with a second visual property.

The first and second visual properties may for example be different shades of grey, colours or intensities. The step of analysing I(ω) may be performed such that a contrast in the processed image is increased between voxels or regions of interest associated with I_{L}(ω_{H}) and voxels or regions of interest associated with I_{S}(ω_{H}).

The inventors have observed that the amplitude I(ω_{H}) in the higher frequency region is often larger for regions of interest which are associated with a flow, such as blood flow in a blood vessel, than for stationary regions. Embodiments of the present invention consequently have the advantage that for example the contrast between blood flow (and thereby blood vessels) and stationary areas of biological tissue can be increased and the identification of blood vessels is consequently improved.

In one embodiment the step of analysing I(ω) may comprise dividing I_{L} (ω_{H}) and I_{S} (ω_{H}) by an amplitude I(ω_{L}) at a frequency ω_{L} in a lower frequency range.

I_{L} (ω_{H}) and I_{S} (ω_{H}) may be respective averages of amplitude within a predetermined frequency range, such as a range of frequencies greater than 0.5, 1, 2 or 3 Hz.

I(ω_{L}) may be an amplitude at a frequency of substantially 0 Hz (DC).

Providing a sequence of at least three images may comprise providing a sequence of at least three depth images. The depth images may be OCT images, such as OCT B-scans each comprising a sequence of OCT A-scans.

Each OCT B-scan may be obtained by detecting a sequence of light spectra (associated with OCT A-scans), and then applying an inverse Fourier transformation to each obtained light spectrum to transform the spectral intensity distribution to a spatial intensity distribution for forming an OCT image (OCT B-scan).

Further, the OCT image may comprise a plurality of OCT B-scans from different locations within the material and which together may form an OCT volume image.

The material may be biological tissue, such as tissue within an eye, such as a human eye, skin or brain. The method is typically performed in-vivo, but may alternatively also be performed ex-vivo.

The invention will be more fully understood from the following description of specific embodiments of the invention. The description is provided with reference to the accompanying drawings.

### BRIEF DESCRIPTION OF DRAWINGS

Notwithstanding any other forms which may fall within the scope of the disclosure as set forth in the Summary, specific embodiments will now be described, by way of example only, with reference to the accompanying drawings in which:

FIG. 1 is a flow chart of a method of detecting a flow in a sequence of images of a material in accordance with an aspect of the present invention;

FIG. 2(a) is a plot of OCT signal frequency magnitude versus frequency obtained for a capillary flow region and a static matrix in the fabricated phantom;

FIG. 2(b) is a plot of OCT signal frequency magnitude versus frequency obtained for a blood vessel and static tissue in the human skin;

FIG. 3(a) is a plot of average high-pass frequency magnitude and contrast as a function of a number of time samples (i.e. co-located B-scans) for a flow region and a static tissue in a phantom in accordance with an embodiment of the present invention;

FIG. 3(b) is a plot of average high-pass frequency magnitude and contrast as a function of a number of time samples (i.e. co-located B-scans) for a flow region and a static tissue in a human skin in accordance with an embodiment of the present invention;

FIG. 4(a) is a cross-sectional vessel image in short-time series OCT Angiography before weighting by the inverse of a mean OCT intensity signal in accordance with an embodiment of the present invention;

FIG. 4(b) is a cross-sectional vessel image in short-time series OCT Angiography (OCTA) after weighting by the inverse of a mean OCT intensity signal in accordance with an embodiment of the present invention;

FIGS. 5(a)-5(b) are OCTA images representing projections of blood vessels obtained by short-time series OCTA based on OCT intensity signal, respectively, before and after weighting by the inverse of a mean OCT intensity signal in accordance with an embodiment of the present invention;

FIG. 5(c) is a projection of blood vessels by short-time series OCTA based on complex OCT signal with weighting.

FIGS. 6(a), (c), and (e) are OCTA images representing projections of blood vessels obtained by, respectively, speckle decorrelation, short-time series, and speckle variance in accordance with specific embodiments of the present invention;

FIGS. 6(b), (d), and (f) are magnifications of regions respectively outlined in FIGS. 6(a), (c), and (e);

FIGS. 7(a), (c), and (e) are further OCTA images representing projections of blood vessels obtained by, respectively, speckle decorrelation, short-time series, and speckle variance in accordance with specific embodiments of the present invention;

FIGS. 7(b), (d), and (f) are magnifications of regions respectively outlined in FIGS. 7(a), (c), and (e);

FIG. 8 is a plot of a normalised OCTA signal as a function of speed in the flow region of a phantom for short-time series, speckle decorrelation and speckle variance, in accordance with an embodiment of the present invention;

FIG. 9(a) is an OCTA image obtained by short-time series representing a projection of blood vessels located between a surface to a depth of approximately 300 μm at a laser-treated skin area of a subject; and

FIG. 9(b) is an OCTA image obtained by short-time series representing a projection of blood vessels located between a surface to a depth of approximately 300 μm at an area of normal skin adjacent to a laser-treated skin area of a subject.

### DETAILED DESCRIPTION OF SPECIFIC EMBODIMENTS

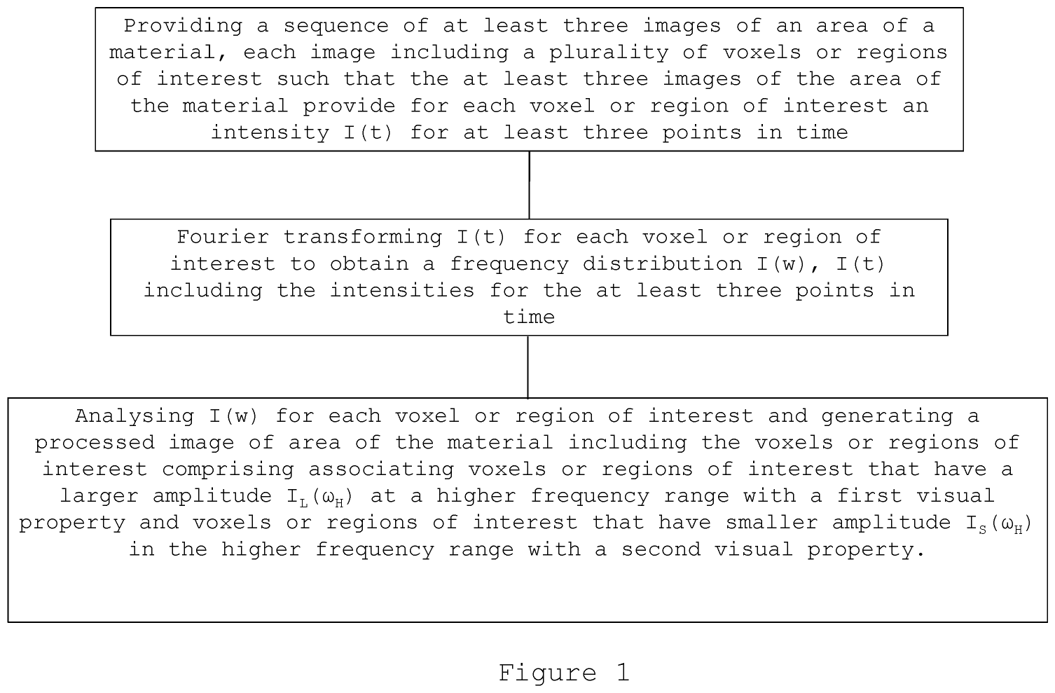

The present invention provides in a first aspect a method of detecting a flow in a sequence of images of a material. Referring to FIG. 1, the method comprises in a first step providing a sequence of at least three images of an area of the material, each image including a plurality of voxels or regions of interest such that the at least three images of the area of the material provide for each voxel or region of interest an intensity I(t) for at least three points in time. In a second step, the method comprises Fourier transforming I(t) for each voxel or region of interest to obtain a frequency distribution I(ω), I(t) including the intensities for the at least three points in time. And, in a third step, the method comprises analysing I(ω) for each voxel or region of interest and generating a processed image of area of the material including the voxels or regions of interest, comprising associating voxels or regions of interest that have a larger amplitude I_{L}(ω_{H}) at a higher frequency range with a first visual property and voxels or regions of interest that have smaller amplitude I_{S}(ω_{H}) in the higher frequency range with a second visual property.

The material in a specific embodiment is a biological tissue and more specifically human skin tissue, is performed in vivo and the method is a method of detecting a flow of blood within a blood vessel in the human skin. In a further embodiment, the material is a fabricated flow phantom comprising capillary regions and a static matrix region, which respectively model blood vessels and static tissue in a human tissue. The method more specifically comprises providing a sequence of at least three depth OCT images.

It will be understood that other biological tissues are however envisaged, such as tissue within a human eye. Materials other than a biological tissue are also envisaged and within the scope of the present invention. Further, the method may be performed ex-vivo.

As mentioned above, the inventors have observed that the amplitude I(ω_{H}) in the higher frequency region is often larger for regions of interest which are associated with a blood flow in a blood vessel than for surrounding static tissue. This finding can be used to image for example blood vessels with a higher contrast.

Improving a blood vessel contrast for each of the flow phantom and for the human skin tissue in vivo will now be discussed. However, a person skilled in the art will appreciate that the present invention has broader applications. Further, it will be understood by a person skilled in the art that the present invention is not limited to OCT, but may be used for magnetic resonant imaging (MRI) and ultrasound imaging (for example).

An embodiment of the present invention comprises taking the frequency spectrum of a detected OCT signal from multiple acquisitions at a given voxel is analysed for each of the flow phantom and the human skin tissue and the method of detecting the flow of blood within a capillary and blood vessel, respectively, is herein referred as a short-time series OCT angiography (OCTA) method. The short-time series OCTA method is also compared to commonly used intensity-based OCTA methods, including speckle decorrelation (correlation mapping) and speckle variance. Results generally demonstrate, for a modest increase in acquisition times for a given OCT A-scan rate in the human tissue, improved vessel contrast and visibility, in particular, for small vessels. Further, the relative simplicity of the method lends itself to fast implementation. These advantages suggest its potential for future applications.

### Methods

### Short-Time Series OCTA Algorithm

The basic assumption underlying the method in accordance with an embodiment of the present invention is that blood flow induces stronger non-zero frequency components in the OCT signal than those induced by the surrounding static tissue. As with other OCTA methods, the method first requires the acquisition of co-located OCT B-scans (i.e., B-scans from the same lateral location) at multiple time points, throughout an acquisition volume. The OCT intensity signal (i.e., the modulus of the complex amplitude of the OCT signal) at the same voxel locations comprises a discrete time series with the nth sample at location (x, y, z) denoted by:

I(x,y,z;tₙ)=I(x,y,z;t₁+(n−1)T), (1)

where (x, y, z) is the voxel coordinate in the fast scanning, slow scanning and depth axes, respectively; I represents the OCT intensity signal as a function of the voxel coordinate with time point tₙ for n, an integer ranging from 1 to 2N+1, where 2N+1 is the total number of co-located B-scans (i.e., total number of time samples) acquired from the same lateral location; and T is the time interval between co-located B-scans.

The time series at each voxel in Equation (1) is discrete Fourier transformed to obtain the complex frequency signal with the frequency components F denoted by:

F(x,y,z;fₘ)=F(x,y,z;mfₒ), (2)

where f₀ is the interval between neighbouring discrete frequencies, determined by 1/[(2N+1)T]; and m is the index of the (two-sided) frequency components ranging from −N to N. The average magnitude of the complex frequency signal at non-zero frequencies is then calculated as

$$F M ( x , y , z ) = \frac{{\sum }_{m = - N , \neq 0}^{N} \vert F ( x , y , z ; {f}_{m} ) \vert}{2 N} . \qquad ( 3 )$$

Alternatively, if many B-scans are acquired for analysis (i.e., 2N+1≥29 for the scanning parameters used in this study), instead of a single frequency component, a narrow band centered on the zero-frequency component is excluded (i.e., high-pass filtered). This narrow band should be optimized for a particular tissue and setup, and will depend on the frequency spectrum recorded from static tissue. The optimization for human skin tissue, recorded using our system parameters, is shown in the Results section. However, there, we demonstrate that only a small number of co-located B-scans (˜5) is required for practical imaging of the vessel network with our method. Thus, the elimination of only the zero-frequency component, as shown in Equation (3), applies.

After Fourier transformation, voxels with low OCT signal intensity lead to a correspondingly low magnitude of the complex non-zero frequency components, even if there is flow. To enhance the flow detectability at low OCT signal levels, we incorporate weighting by the inverse of the OCT signal intensity (i.e., zero-frequency component scaled by the number of co-located B-scans), given by:

$$F {M}_{W} ( x , y , z ) = \frac{F M ( x , y , z )}{\overline{I ( x , y , z )}} = \frac{2 N + 1}{2 N} \frac{ \begin{matrix}{\sum }_{m = - N , \neq 0}^{N} \\ \vert F ( x , y , z ; {f}_{m} ) \vert\end{matrix}}{\vert F ( x , y , z ; 0 ) \vert} , \qquad ( 4 )$$

where I̅(̅x̅,̅y̅,̅z̅)̅ is the mean OCT signal intensity and |F(x,y,z;0)| is the zero-frequency component of the 2N+1 time samples at the same location. To avoid division by zero and over-emphasizing the signal in regions with excessive noise, I̅(̅x̅,̅y̅,̅z̅)̅ is first averaged, and thresholded at an empirically chosen signal level of 16 dB above the noise floor to replace the low signal with the threshold. We used an averaging window of 3×3 pixels in the cross-sectional plane, approximately 1.4 and 1.9 times the lateral and axial resolutions, respectively. It is used both for our method and for the accompanying speckle decorrelation calculation, empirically chosen to improve the signal-to-noise ratio (SNR) of angiography without significantly degrading the imaging resolutions. We assume that an odd number of co-located B-scans is acquired for each lateral location for simplicity, but even numbers are applicable as well. The vessel contrast created by Equation (3) and the further improvement introduced by weighting according to Equation (4) will be demonstrated and discussed in the Results section.

### OCT Scanning of Flow Phantom and Human Skin

OCT scans were acquired using a commercial spectral-domain scanner (an upgraded TELESTO II, Thorlabs Inc., USA) to demonstrate the short-time series OCTA method on both a flow phantom and in vivo on normal human skin. The system has a center wavelength of 1300 nm and provides an imaging resolution of 5.5 μm (in air) and 13 μm, respectively, axially and laterally (as defined by the vendor). The scanner was operated at 76 kHz (A-scan/s), below its maximum of 146 kHz. Scans were acquired in one of two modes: 2D scanning by acquiring 200 co-located B-scans from a single lateral location with a FOV of 6×3.6 mm (1024×1024 pixels) in x and z directions, respectively; and 3D scanning with a FOV of 6×1.8×3.6 mm in x, y and z directions. In 3D scanning mode, 240 lateral (y) locations were scanned with a set of 5 co-located B-scans acquired from each location, using the same pixel sizes in x and z directions as in the 2D mode. It took approximately 4 and 21 s to acquire a scan, respectively, in the 2D and 3D scanning modes. In addition, the time interval between B-scans was 17.8 ms (˜56 B-scans/s) for both 2D and 3D modes, leading to a discrete frequency spectrum with components up to 28 Hz.

For comparison to short-time series OCTA, speckle variance in the same 3D scans was calculated over the 5 co-located B-scans using the method presented by Mariampillai et al. in “Speckle variance detection of microvasculature using swept-source optical coherence tomography,” Opt. Lett. 33(13), 1530-1532 (2008). Speckle decorrelation was determined for each adjacent pair of co-located B-scans using the formula described in P. Gong, S. Es'haghian, K. A. Harms, A. Murray, S. Rea, B. F. Kennedy, F. M. Wood, D. D. Sampson, and R. A. McLaughlin, “Optical coherence tomography for longitudinal monitoring of vasculature in scars treated with laser fractionation,” J. Biophotonics 9(6), 626-636 (2016), with a window of 3×3 pixels in the fast scanning and depth axes. This led to four decorrelation B-scans from each lateral location, which were then averaged to generate a single enhanced decorrelation B-scan. In addition, the speckle decorrelation and speckle variance was weighted by the averaged and thresholded OCT signal at the corresponding pixels to reduce the noise (J. Enfield, E. Jonathan, and M. Leahy, “In vivo imaging of the microcirculation of the volar forearm using correlation mapping optical coherence tomography (cmOCT),” Biomed. Opt. Express 2(5), 1184-1193 (2011)). The threshold used in short-time series OCTA was used here. The same lateral averaging window (3×3 pixels) was applied to the short-time series and speckle variance images to ensure a fair comparison.

Blood vessels were mainly compared over a depth range of 300 μm from the skin tissue surface (determined from the OCT depth scan by assuming an average refractive index of 1.4) to ensure sufficiently strong signals from all three methods. For each method, the maximum OCTA signal of each A-scan in this depth range was used to generate a projection image of vessels. For visualization, the same colormap was used in the projection and cross-sectional OCTA images. The lower and upper thresholds were set at, respectively, the 50% and 99.5% points of the cumulative distribution function of the OCTA signal in the image. These thresholds were empirically chosen to maximize the vessel contrast without loss of vessels with low signal. For quantification, each projection image was processed to measure the vessel area density, defined as the ratio of the total vessel area to the total tissue area in the thresholded vessel image. The threshold was set using the Otsu's method for each image (N. Otsu, “A threshold selection method from gray-level histograms,” IEEE Trans. Syst. Man Cybern. 9(1), 62-66 (1979)).

The silicone flow phantom was fabricated in house by mixing Elastosil® P7676A and P7676B fluid (Wacker Chemie AG, Germany) with titanium dioxide in a 3D-printed plastic container (see, S. Es'haghian, K. M. Kennedy, P. Gong, D. D. Sampson, R. A. McLaughlin, and B. F. Kennedy, “Optical palpation in vivo: imaging human skin lesions using mechanical contrast,” J. Biomed. Opt. 20(1), 016013 (2015)). The container was customized with two holes in the sidewall to hold a small glass capillary (outer diameter: 80 μm; inner diameter: 50 μm) that mimicked a blood vessel. After curing, the capillary was embedded in the silicone that mimicked the static tissue. The capillary was then connected to a syringe filled with a polystyrene microsphere suspension (nominally 0.5-μm diameter) to mimic the blood flow. The syringe was connected to a pump (Fusion 200, Chemyx Inc., USA) to introduce and control the flow speed. The scattering properties of the phantom were adjusted by tuning the ratio of titanium dioxide to Elastosil® P7676A and P7676B so that the phantom had a signal attenuation that approximately matched the attenuation of normal human skin.

Human subjects (n=4) were recruited for in vivo OCT scanning with ethics approval from the Human Research Ethics Committee of The University of Western Australia. Written consent was acquired from all subjects prior to OCT scanning of skin on the volar forearm, including one subject who had received laser treatment for wart removal. For this subject, one region from the treated area and one from the adjacent normal skin were selected for OCT imaging. To reduce bulk tissue motion during data acquisition, a spacer was attached to the skin surface to tightly couple the OCT probe and the skin tissue. A piece of thin metal with a center hole (5 mm in diameter) to image through was also attached to the skin as a fiducial marker to check for motion artefact. We observed a generally good vessel contrast and negligible vessel distortions, so no motion correction was performed. Further details on the imaging probe spacer and scanning setup can be found in P. Gong et al., “Optical coherence tomography for longitudinal monitoring of vasculature in scars treated with laser fractionation,” J. Biophotonics 9(6), 626-636 (2016). The acquired scans from the phantom and skin tissue were then processed using the three OCTA methods and compared.

### Results

This section firstly considers the contrast present in the short-time series OCTA method and its optimization by selecting the number of samples and incorporating signal weighting. The difference between short-time series implemented on the OCT intensity and on the complex signal is also shown. Results from the optimized short-time series OCTA method are then compared to those acquired from speckle decorrelation and speckle variance.

### Vessel Contrast

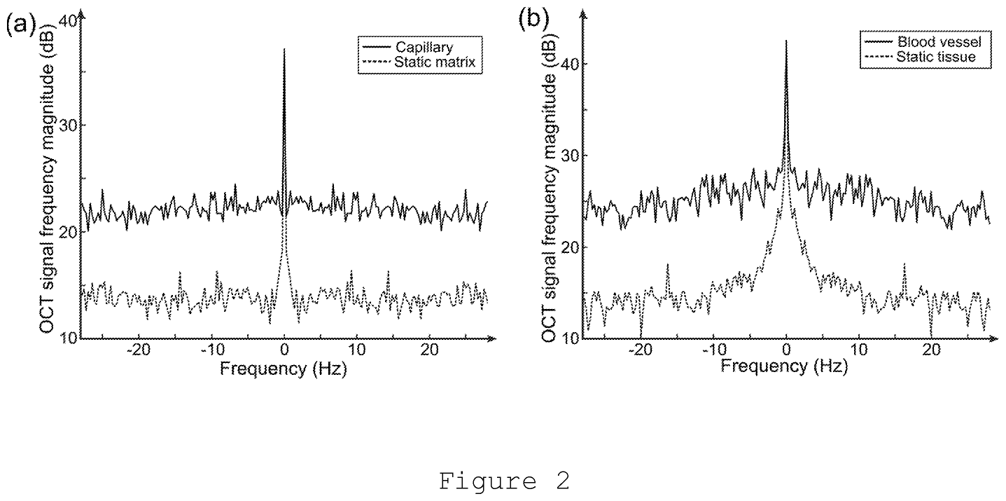

The blood vessel contrast in short-time series OCTA originates from the elevated non-zero frequency components induced by the moving scatterers in blood. An example of such vessel contrast, obtained from an extended time series of 200 B-scans,is shown in FIG. 2(a), which plots the Fourier transform (magnitude) of the time series of the OCT intensity signal in the phantom scan across polystyrene microsphere flow in a capillary.

Two-sided spectral density of the signal from 200 B-scans in the flow and static regions is shown in FIG. 2(a) for the phantom and FIG. 2(b) for human skin tissue.

The frequency spectrum of the OCT signal from the static matrix region in FIG. 2(a) is ˜20 dB lower at ˜1.1 Hz than its value at the peak and remains consistently low for frequencies above this. Whilst a similar sharp drop-off is observed in the capillary flow region (for flow speed 3 mm/s), the magnitude for frequencies at and above 1.1 Hz is much higher (FIG. 2(a)) than in the static matrix.

Similar plots were obtained from in vivo skin, determined from 200 co-located B-scans, with the spectral density shown in FIG. 2(b), indicating consistent contrast between blood vessels and static tissue. This contrast may be parameterized as the average magnitude of the non-zero (high-pass) frequencies using Equation (3). In addition, FIG. 2(b) indicates that the contrast between flow and static tissue is present at frequencies higher than ˜2 Hz, which is chosen as the cut-off for the calculation of average magnitude at high frequencies in Equation (3). Note that the peak centered at zero frequency for the static tissue in skin (FIG. 2(b) has a larger width than that of the static matrix in the phantom (FIG. 2(a)). This may be due to residual motion in the skin tissue, from the pulse (approximately 60-100 beats per minute) or from other sources. In the following sections, when only 5 co-located B-scans are acquired for analysis, the frequency interval is much larger (i.e., 11.2 Hz), requiring only removal of the zero-frequency component, as per Equation (3), to achieve the desired high-pass filtering.

### Choice of Time Series Length

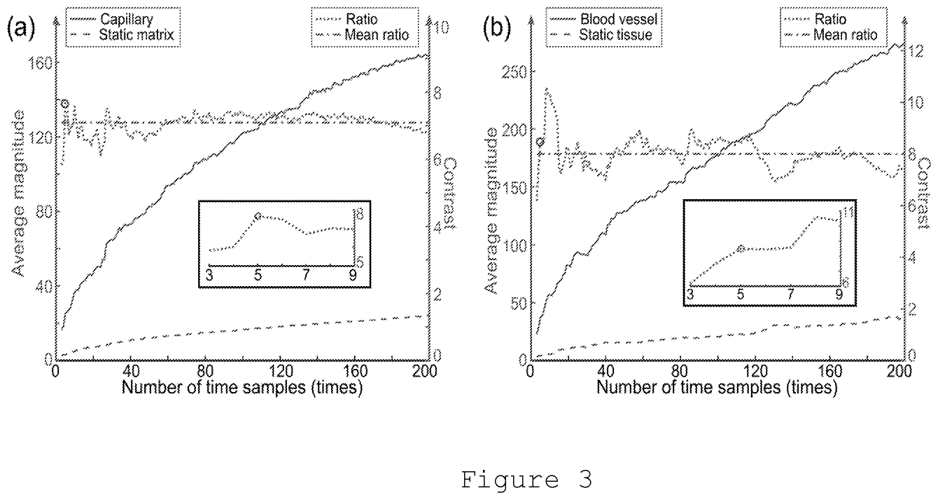

The frequency spectra shown in FIG. 2 are from acquisitions comprising 200 co-located B-scans, chosen to enable detailed analysis at a single lateral location. Such long acquisitions are not practical for clinical applications: a trade-off is needed to reduce the number of time samples whilst maintaining high vessel contrast. The inventors investigated this trade-off, presented in FIG. 3, showing average magnitude and contrast obtained by calculating the average magnitude for frequencies above 2 Hz for the flow region (solid) and static tissue (dashed) comprising 3×3 pixels in the phantom (FIG. 3(a)) and skin tissue (FIG. 3(b)). FIG. 3 indeed illustrates vessel contrast in time series OCTA for varying numbers of time samples (i.e., co-located B-scans) in the phantom FIG. 3(a) and skin tissue FIG. 3(b). The average high-pass frequency magnitude versus number of time samples is shown for the flow (solid) and static tissue regions (dashed) with reference to the left vertical axis. Their ratio is shown by the dotted plots with the mean value marked by the dashed-dotted line relative to the right vertical axis. Insets show the magnified traces of contrast for 3-9 samples. The circles indicate the ratios for 5 samples.

The average magnitude increases, for both the flow regions in the capillary/blood vessels and for the static matrix/static tissue, versus the number of acquired co-located B-scans, as does the difference in the average magnitude between the flow and static regions. Notably, the ratio between the two (dotted plots) peaks at around 5-10 co-located B-scans before it reaches a plateau (with local fluctuations). In FIGS. 3(a) and (b), the dashed-dotted lines show the mean of all the ratios (n=3 to 200), with a value in the range 7-8. The circles show the ratio for 5 B-scans. This figure indicates that acquisition of ˜5 co-located B-scans is sufficient to provide a good vessel contrast with the short-time series method. Acquiring more co-located B-scans may be beneficial in some circumstances but increases acquisition time. In contrast, acquiring only 3 or 4 co-located B-scans still shows clear contrast for the vessel analyzed in FIG. 3(b), but can be problematic for vessels with lower contrast. We, therefore, chose to use 5 co-located B-scans from the same tissue location for the 3-D imaging of the vessel network presented below.

### Signal Enhancement by Weighting

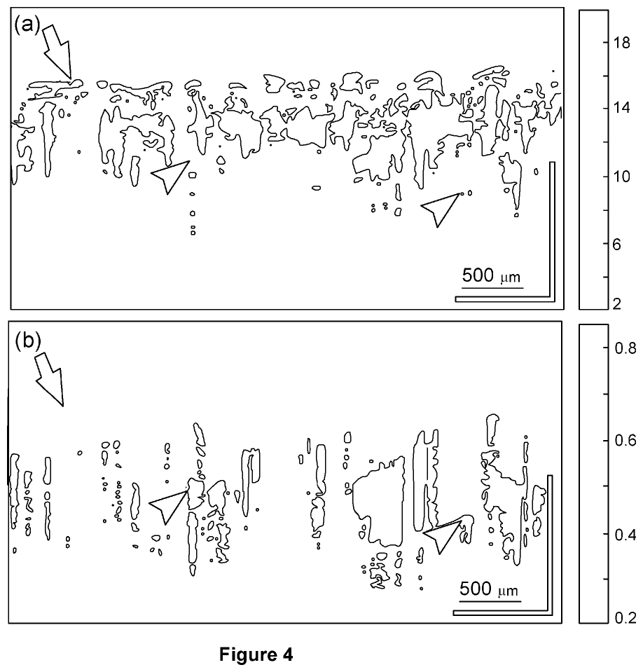

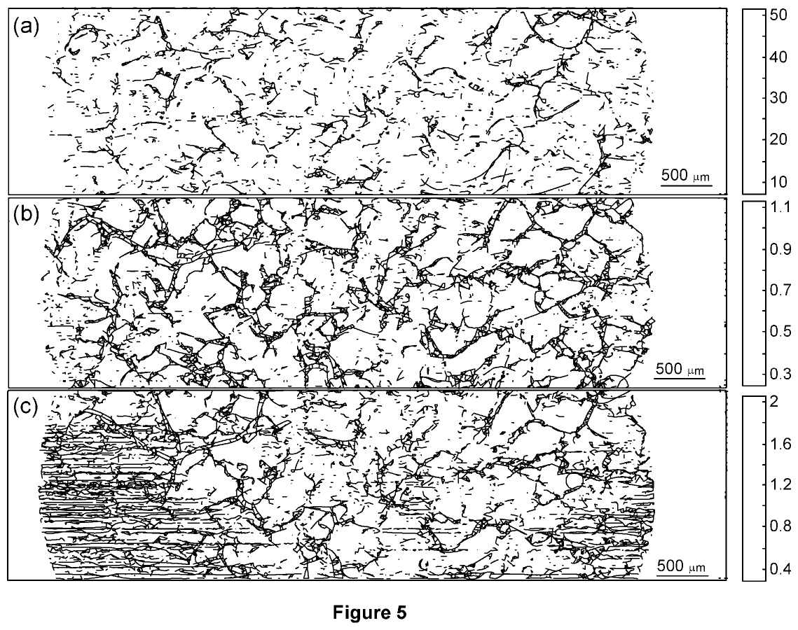

To enhance the vessel signal in the flow regions with low OCT signal, we further weight the average magnitude of non-zero frequencies by the inverse of the linear OCT signal intensity, as described in Equation (4). The weighted image in FIG. 4(b) shows increased vessel signal (e.g., for vessels marked by the two arrowheads), compared to FIG. 4(a) before weighting. The level of improvement is better appreciated in the projection images, comprising vessels from the forearm skin surface to 300 μm deep, as shown in FIG. 5(b). Compared to the equivalent unweighted image in FIG. 5(a), the weighted image shows improved visibility in terms of vessel connectivity and the number of visible vessels. In addition, the weighting significantly suppresses the artificial vessel signals caused by very strong surface reflections, as indicated by the arrow in FIG. 4(a). Therefore, we incorporate this weighting into our method as an important step in optimizing the vessel image quality.

FIG. 4 illustrate a cross-sectional vessel image in short-time series OCTA before (a) and after (b) weighting by the inverse of the mean OCT intensity signal. Arrows and arrowheads indicate the corresponding pixels in the two images at the tissue surface (arrows) and blood vessels (arrowheads), respectively. The scale bars correspond to 500 μm.

FIG. 5 illustrate a projection of blood vessels by short-time series OCTA based on OCT intensity signal before (a) and after weighting (b), and the complex OCT signal with weighting (c). The projections display vessels to 300 μm deep from the skin surface. The scale bars correspond to 500 μm.

Intensity Vs. Complex Signal-Based Processing

As with other OCTA methods, it is possible to analyze either the intensity or the full complex OCT signal (i.e., intensity and phase). The short-time series method was applied to both cases in the same skin scans. A representative example is shown by the vessel projection images in FIG. 5. A comparison of FIGS. 5(b) and 5(c) indicates that intensity and complex signal-based processing lead to a very comparable detection of vessels. However, using the complex signal produces more motion artefacts (horizontal lines) evident in FIG. 5(c).

A customized imaging spacer and setup was used to minimize motion during data acquisition, which has previously been shown to be effective when used in combination with the speckle decorrelation method (P. Gong et al., “Optical coherence tomography for longitudinal monitoring of vasculature in scars treated with laser fractionation,” J. Biophotonics 9(6), 626-636 (2016)).

Whilst residual tissue motion is almost absent in FIGS. 5(a) and (b), based on intensity only, it is still detectable as multiple horizontal lines in FIG. 5(c), due to the incorporation of the more motion-sensitive phase information. An additional motion correction algorithm would be required to mitigate such artefacts, if the complex signal was to be the basis of the method. To avoid this need, the short-time series OCTA results in the following section were calculated using the OCT intensity signal alone.

Comparison with Speckle Decorrelation and Speckle Variance

To further assess the performance of the OCTA method in accordance with embodiments of the present invention, the short-time series OCTA method was compared to two commonly used intensity-based OCTA methods, speckle decorrelation (correlation mapping) and speckle variance, applied to sets of 5 co-located B-scans in 3D scans.

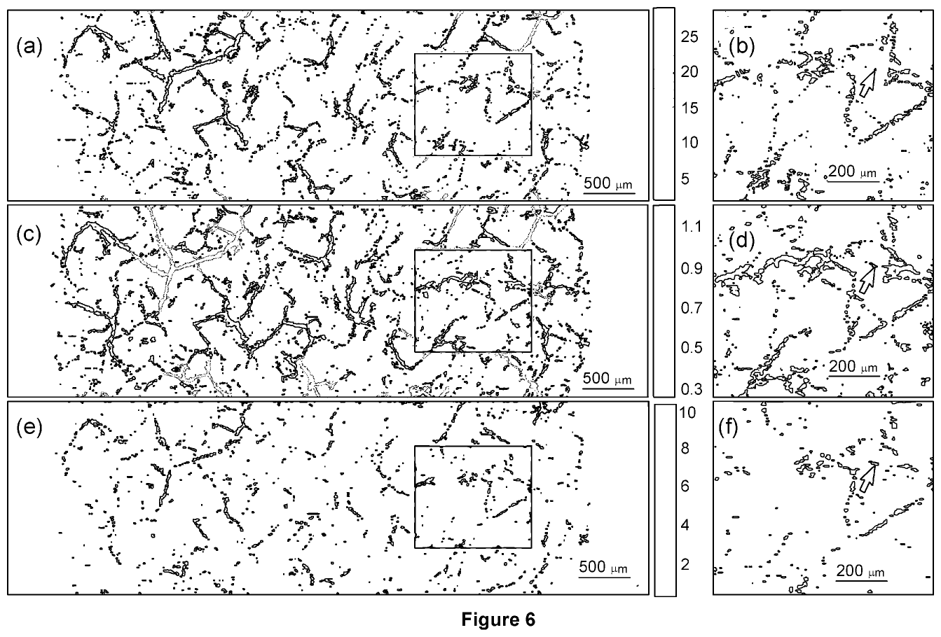

Referring to FIG. 6, there is shown in FIG. 6(a) a projection of blood vessels by speckle decorrelation, in FIG. 6(c) a projection of blood vessels by short-time series and in FIG. 6(e) a projection of blood vessels by speckle variance. The outlined regions in FIGS. 6(a), 6(c) and 6(e) are magnified in FIGS. 6(b), 6(d) and 6(f), respectively.

Vessels from the forearm skin are projected from the surface to 300 μm in depth. The arrows in the FIG. 6 mark the same vessel segment. The scale bars correspond to 500 μm in (a), (c) and (e), and to 200 μm in (b), (d) and (f).

Thus, FIG. 6 shows an example from forearm skin, projecting the blood vessels from the skin surface to 300 μm in depth. Vessel images generated by the short-time series method are in the middle row to allow easy comparison to images generated by speckle decorrelation (above) and speckle variance (below). In FIG. 6(c), the short-time series method provides visualization of the vessel network that is comparable to the speckle decorrelation (FIG. 6(a)) and speckle variance methods (FIG. 6(e)). Further examination indicates the improved contrast of the blood vessels in the short-time series projection image, observed as the enhanced connectivity and visibility of the vessels. One such example, taken from the outlined tissue regions in FIG. 6(c), is magnified in FIG. 6(d). In comparison to FIGS. 6(b) and 6(f), obtained using speckle decorrelation and speckle variance, respectively, several vessel segments are more clearly observed, with a representative example marked by the arrows.

Such improvement is further quantified by measuring the vessel area density, with an estimated accuracy of approximately 1%. This results in a superior area density of 28% for the short time-series image shown in FIG. 6(c), in comparison to 21% and 20% for speckle decorrelation and speckle variance, respectively. The higher density in short time-series method results from the improved vessel contrast, as shown in FIG. 6.

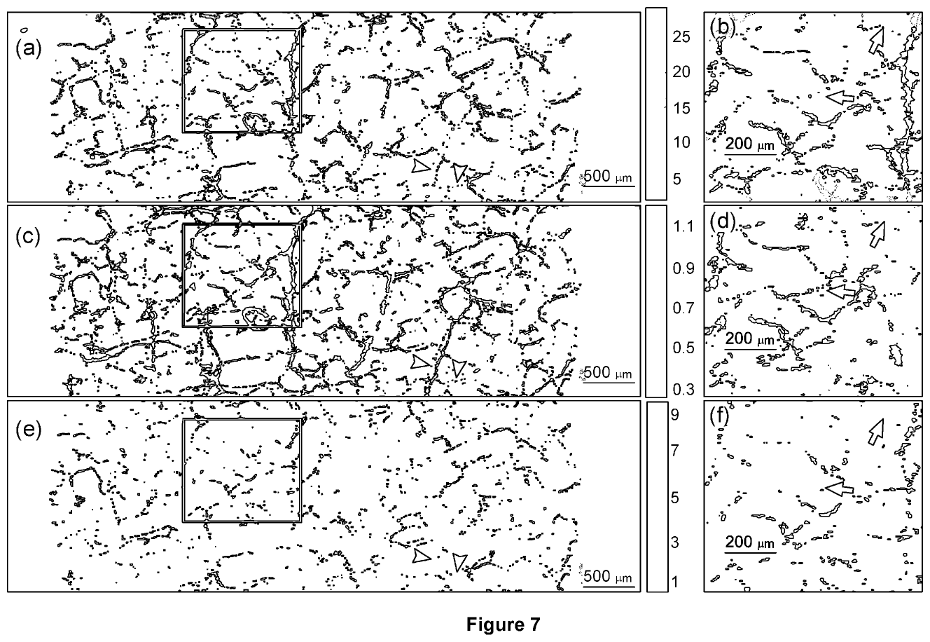

The consistent superiority of vessel contrast afforded by short time series OCTA is observed in all human subjects (n=4) in this study, with a further example shown in FIG. 7. FIG. 7(a) is a projection of blood vessels by speckle decorrelation, FIG. 7(c) is a projection of blood vessels by short-time series and FIG. 7(e) is a projection of blood vessels by speckle variance. The outlined regions in FIGS. 7(a), 7 (c) and 7 (e) are magnified in FIGS. 7(b), 7 (d) and 7(f), respectively. Vessels from the forearm skin are projected from the surface to 300 μm in depth. The arrowheads in the (a), (c) and (e) mark the same vessel segments. The arrows in the (b), (d) and (f) mark the same vessel segments. The scale bars correspond to 500 μm in (a), (c) and (e), and to 200 μm in (b), (d) and (f). The maximum intensity projection of the blood vessels, from the skin surface to 300 μm deep by the three methods, is visualized in FIGS. 7(a), 7(c) and 7(e). This case also shows the superior vessel visibility provided by the short time-series method in FIG. 7(c), over speckle decorrelation (FIG. 7(a)) and speckle variance (FIG. 7(e)). The vessel contrast differences are highlighted by the corresponding magnified region in FIGS. 7(b), 7(d) and 7(f), indicating the superior vessel signal strength and connections evident in the short-time series images, with specific instances marked by the arrows. The measured vessel area density (27%), in this case, is higher than for speckle decorrelation (21%) and speckle variance (19%), consistent with the analysis in FIG. 6. Interestingly, this case shows several examples of parallel vessels in local regions (e.g., the vessels marked by the arrowheads), which are easier to appreciate in the short-time series images than in the images obtained by speckle decorrelation or speckle variance. Another projection approach used in the literature is to take the mean OCTA vessel signal, instead of the maximum (see, e.g., C. L. Chen, and R. K. Wang, “Optical coherence tomography based angiography [Invited],” Biomed. Opt. Express 8(2), 1056-1082 (2017), and A. Zhang et al., “Minimizing projection artifacts for accurate presentation of choroidal neovascularization in OCT micro-angiography,”Biomed. Opt. Express 6(10), 4130-4143 (2015)). In this study, consistent vessel contrast differences among the three methods are observed in the mean projections as well (not shown).

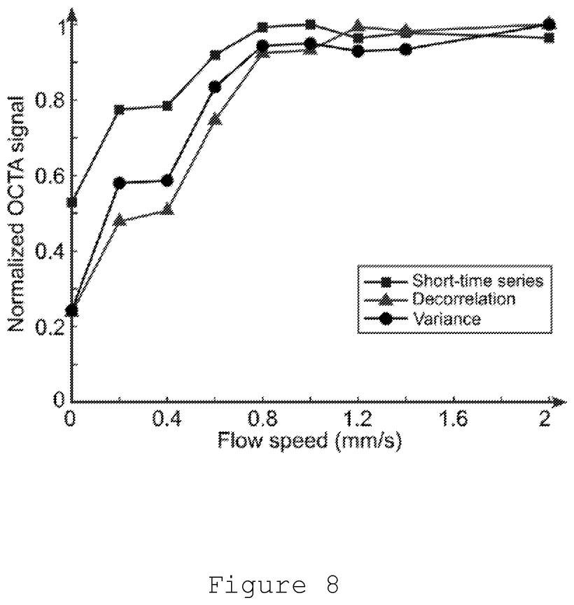

To further elucidate the contrast differences among the three methods, an experiment was performed to examine the OCTA signal in the phantom versus flow speed (9 values ranging from 0 to 2 mm/s). FIG. 8 shows a normalized OCTA signal versus flow speed in the flow region of the phantom for short-time series (squares), speckle decorrelation (triangles) and speckle variance (circles). Specifically, FIG. 8 shows the resulting signal strength in the flow region, determined by subtracting the noise in the static region from the original flow signal and then normalizing the flow signals to their maximums. All three methods show an increase of the signal strength with increasing flow speed of the microspheres (diameter: 2 μm), from the baseline signal due to Brownian motion at zero flow speed. The signals then all saturate at approximately 0.8-1.2 mm/s. Compared to speckle decorrelation and speckle variance, the short-time series method shows higher normalized signal in the low speed range. This observation is consistent with the improved contrast for small vessels seen in FIGS. 6 and 7.

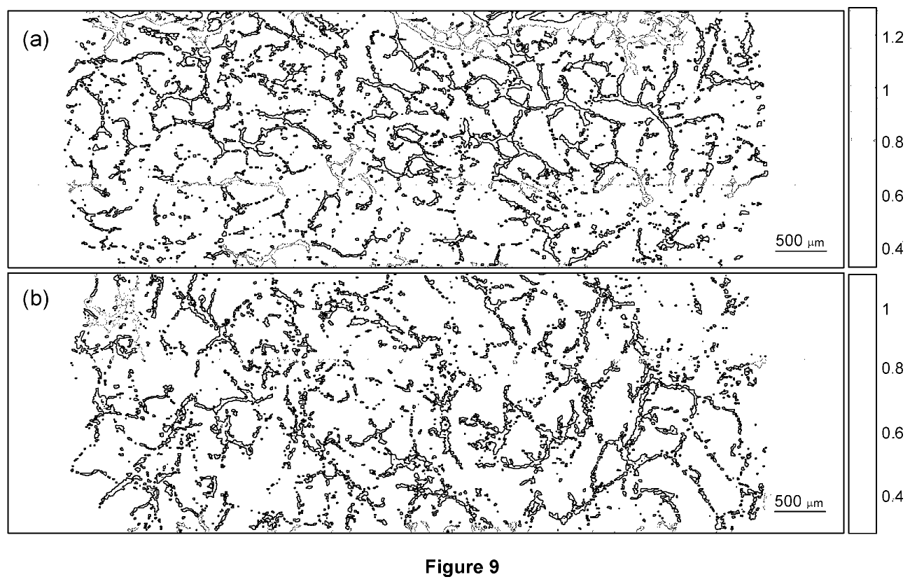

In addition to visualization of normal vessel networks, short-time series OCTA also shows good vessel contrast for the subject with a treated wart. The resulting vessel image is shown in FIG. 9(a) in comparison to the adjacent normal skin of the same subject in FIG. 9(b). FIG. 9 specifically illustrate short-time series OCTA imaging of a subject with a laser-treated wart, wherein FIG. 9(a) shows a projection of blood vessels from the surface to 300 μm in depth of the laser-treated area and FIG. 9(b) shows a projection of blood vessels from the surface to 300 in depth of the adjacent normal skin (b). The scale bars correspond to 500 μm.

The wart was removed with a laser ˜16 years prior to OCT scanning. Comparison of the images generated by the three OCTA methods consistently shows the improved visualization by the short-time series method, for both the normal and treated skin regions (not shown). Though the treated region shows a very comparable skin color to the normal skin, the underlying microvasculature visualized by the short-times series method clearly reveals the morphological differences. For example, the treated region presents a network with more branches and a distinct honeycomb-like pattern (i.e., local loops), absent from the normal skin. The quantified vessel area density in the treated region (34%) is significantly higher than that in the normal skin (29%). Such visualization and the associated contrast demonstrate the potential of short-time series OCTA for future studies of various cutaneous conditions.

Another important factor is the computation time, which can be limiting for applications requiring near-real-time or real-time imaging. Overall, speckle decorrelation is more time consuming than short-time series or speckle variance due to the requirement for window processing (not simply averaging) to generate the vessel signal. It took ˜420 ms to calculate the decorrelation of a pair of B-scans (1024×1024 pixels per B-scan) using a 3×3 pixel window on an Intel® Core™ i7-3820 processor with MATLAB R2016a (The MathWorks, Inc.). When a larger window is used for processing, the computation time increases significantly (e.g., 990 ms for a 5×5 pixel window). In contrast, data processing for the short-time series method and speckle variance is much faster, taking ˜64 ms and ˜27 ms, respectively, to process each set of 5 co-located B-scans. This feature indicates the promise for future implementation of the short-time series method on fast scanning OCT systems to enable in-procedure or even real-time visualization of microvasculature.

### DISCUSSION

The method proposed in accordance with the described embodiment takes a short time series of OCT B-scans, i.e. a sequence of at least three images acquired at the same location as an input, and performs a discrete Fourier transform to determine the frequency content in order to image blood vessels. The observed higher magnitudes at non-zero (high-pass) frequencies in the blood vessels (up to 28 Hz demonstrated here) create a clear contrast to distinguish blood vessels from surrounding static tissue. This method is easily applicable to OCT scans acquired using normal scanning parameters for imaging of the microvascular network. In case studies on human skin, short-time series OCTA shows moderately but consistently improved vessel contrast in comparison to speckle decorrelation and speckle variance, especially for the smaller vessels. Whilst the in vivo comparison was demonstrated on skin tissue, application of short-time series OCTA method to other biological tissues, such as the retina, is also envisaged.

The number of co-located B-scans acquired from the same location is an important parameter for the practical implementation of the short-time series method in accordance with embodiments of the present invention. We chose five in this study so as to minimize the amount of collected data and corresponding total acquisition time, whilst still attaining a high vessel/static tissue contrast in skin.

Thus, the short-time series OCTA method in accordance with the specific embodiment of the present invention demonstrates the performance of imaging of tissue microvasculature in vivo, wherein the flow-induced signature in the frequency domain via Fourier transform of the time series of the OCT signal in five B-scans from the same lateral location was analysed. The angiography signal is computed as the average magnitude of the non-zero (high-pass) frequency components, clearly differentiating blood vessels and static tissue, as demonstrated in a flow phantom and in human skin in vivo. Weighting of the angiography signal by the inverse of the mean OCT signal demonstrated improved detection of blood vessels. The imaging performance of short-time series OCTA was assessed by comparison to the commonly used speckle decorrelation and speckle variance methods, showing consistently substantially improved results, evidenced by improved visualization, especially for small vessels, and increased vasculature density of the human cutaneous microvascular network.

It is also to be understood that, if any prior art publication is referred to herein, such reference does not constitute an admission that the publication forms a part of the common general knowledge in the art, in Australia or any other country.

1-14. (canceled)

15. A method of detecting a flow in a sequence of images of a material, the method comprising the steps of:
providing a sequence of at least three images of an area of the material, each image including a plurality of voxels or regions of interest such that the at least three images of the area of the material provide for each voxel or region of interest an intensity I(t) as a function of time t for at least three points in time;
Fourier transforming I(t) for each voxel or region of interest to obtain a distribution I(ω) of frequency co, I(t) including the intensities for the at least three points in time; and
analysing I(ω) for each voxel or region of interest and generating a processed image of the area of the material including the voxels or regions of interest, comprising associating voxels or regions of interest that have a larger amplitude I_{L}(ω_{H}) at a frequency ω_{H} in a higher frequency range than other voxels or regions of interest with a first visual property and voxels or regions of interest that have smaller amplitude I_{S}(ω_{H}) in the higher frequency range than other voxels or regions of interest with a second visual property;
wherein the larger amplitude I(ω_{H}) is associated with a flow and the smaller amplitude I_{S}(ω_{H}) is associated with a stationary region.

16. The method of claim 15 wherein the flow is a flow of blood in a blood vessel.

17. The method of claim 15 wherein the first and second visual properties are different shades of grey, colours or intensities.

18. The method of claim 16 wherein the first and second visual properties are different shades of grey, colours or intensities.

19. The method of claim 15 wherein the step of analysing I(ω) is performed such that a contrast in the processed image is increased between voxels associated with I_{L}(ω_{H}) and voxels or regions of interest associated with I_{S}(ω_{H}).

20. The method of claim 16 wherein the step of analysing I(ω) is performed such that a contrast in the processed image is increased between voxels associated with I_{L}(ω_{H}) and voxels or regions of interest associated with I_{S}(ω_{H}).

21. The method of claim 17 wherein the step of analysing I(ω) is performed such that a contrast in the processed image is increased between voxels associated with I_{L}(ω_{H}) and voxels or regions of interest associated with I_{S}(ω_{H}).

22. The method of claim 18 wherein the step of analysing I(ω) is performed such that a contrast in the processed image is increased between voxels associated with I_{L}(ω_{H}) and voxels or regions of interest associated with I_{S}(ω_{H}).

23. The method of claim 20 wherein the method comprises generating the processed image with improved blood vessel contrast.

24. The method of claim 15 wherein the step of analysing I(ω) comprises dividing I_{L}(ω_{H}) and I_{S}(ω_{H}) by an amplitude I(ωL) at a frequency ω_{L} in a lower frequency range.

25. The method of claim 15 wherein I_{L}(ω_{H}) and I_{S}(ω_{H}) are respective averages of amplitudes within a predetermined frequency range, such as a range of frequencies greater than 0.5, 1, 2 or 3 Hz.

26. The method of claim 15 wherein I_{L}(ω_{L}) is an amplitude for a frequency of substantially zero (DC).

27. The method of claim 15 wherein providing a sequence of at least three images comprises providing a sequence of at least three depth images.

28. The method of claim 27 wherein the depth images are OCT images, such as OCT B-scans comprising a sequence of OCT A-scans.

29. The method of claim 28 wherein the OCT image may comprise a sequence of OCT B-scans from different locations within the material in order to obtain a volume image.

30. The method of claim 15 wherein providing a sequence of at least three images comprises obtaining OCT light spectra and then applying an inverse Fourier transformation to each obtained OCT light spectrum to transform the spectral intensity distribution associated with the OCT A-scan to a spatial intensity distribution for forming an image.

31. The method of claim 15 wherein the material is biological tissue, such as tissue within an eye and skin, such as a human eye and skin.

32. The method of claim 16 wherein the material is biological tissue, such as tissue within an eye and skin, such as a human eye and skin.

33. The method of claim 15 wherein the method is performed in-vivo.

34. The method of claim 16 wherein the method is performed in-vivo.
